(19) United States
(12) Patent Application Publication
(10) Pub. No.: US 2008/0287541 A1
(43) Pub. Date: Nov. 20, 2008

(54) Kit for Treatment of Cancer

(75) Inventors: Arnold Hoffman, Rehovot (IL); Lee M. Spetner, Jerusalem (IL); Michael Burke, Ramat Gat (IL)

Correspondence Address: BROWDY AND NEIMARK, P.L.L.C.;624 NINTH STREET, NW, SUITE 300, WASHINGTON, DC, 20001-5303, US

(73) Assignee: Redoxia Israel Ltd., Jerusalem (IL)

(21) Appl. No.: 11/596,043

(22) PCT Filed: May 9, 2005
(86) PCT No.: PCT/IL05/00490

§ 371 Date: Aug. 15, 2007

(30) Foreign Application Priority Data
May 10, 2004 (IL) 161899

(51) Int. Cl. A61K 31/195; A61P 35/00

(52) U.S. Cl. 514/562

(57) ABSTRACT

The present invention relates to a kit for the treatment of cancer comprising (a) a container for containing a first compound (i) or a precursor thereof, said first compound or precursor being a compound that oxidizes glutathione (GSH); (b) a container for containing a second compound (ii) or a precursor thereof, said second compound or precursor being a compound that forms an adduct or conjugate with GSH; (c) a container for containing a third compound (iii) or a precursor thereof, said third compound or precursor being a compound that inhibits the rate-limiting enzyme of GSH biosynthesis, gamma-glutamylcysteine synthetase (GCS); and (d) a container for containing a fourth compound (iv) or a precursor thereof, said fourth compound or precursor being a compound that inhibits the enzyme responsible for the conversion of GSSG to GSH, glutathione reductase (GR).

### FIELD OF THE INVENTION

The present invention relates to kits and methods for the treatment of cancer, in particular by altering the redox state or environment of the cell, more particularly by altering the balance of GSH (glutathione) to GSSG (glutathione disulfide), and continuously maintaining this altered state for an appropriate time duration.

ABBREVIATIONS: BCNU: 1,3-bis-(2-chloroethyl)-1-nitrosourea; BSO: buthionine sulfoximine; Carmustine: BCNU; CCP: cessation of cell proliferation; E: intracellular redox potential; GCL: γ-glutamylcysteine synthetase; GCS: GCL; GR: glutathione reductase; GS: glutathione synthetase; GSH: glutathione; GSSG: glutathione disulfide; RB: retinoblastoma protein; ROS: reactive oxygen species.

### BACKGROUND OF THE INVENTION

The redox state of a cell refers to the balance between oxidative processes and reducing processes. The energy released by oxidative processes is used by the cell to build cellular and tissue structures, and to operate and maintain such structures. The term redox state has typically been used to refer to two molecules between which electrons may be traded, and which are referred to as a “redox couple”. An example of such a couple is made up of the two molecules glutathione (GSH) and its oxidized form, glutathione disulfide (GSSG), which help to determine the balance between oxidative and reducing processes, and hence the redox state or environment of the cell. Another redox couple comprises NADPH and NADP⁺. The balance between the oxidized and reduced forms of these couples may have many important biological effects, particularly with regard to the growth and proliferation of the cell.

Without wishing to rule out other mechanisms, it can be assumed that the redox state of the cell has some measure of control over the proliferative behavior of the cell, and in particular to the induction of cessation of cell proliferation (CCP), as explained in greater detail below.

One way to describe the redox state or environment of the cell is through the Nernst equation. Changes in the intracellular redox potential, E, are, according to the Nernst equation, proportional to changes in log {[GSH]²/[GSSG]}, where [GSH] and [GSSG] are the concentrations of GSH and GSSG, respectively. As [GSH] decreases, E increases (Hutter et al., 1997).

Decreasing the level of GSH increases the redox potential of the cell, and has been observed to lower the rate of cell proliferation. Normal actively proliferating (foreskin) fibroblasts have been observed to have an average E of about −222 mV, which is about 10 mV lower than that observed for neoplastic fibrosarcoma cells, where the average E has been observed to be about −211 mV (Hutter et al., 1997). Proliferative behavior appears to be associated with the redox potential of the cell. Decreasing the level of GSH increases the redox potential of a cell, and has been shown to result in a decrease, or cessation of, cell proliferation. Again, without limiting the process to a single mechanism, we suggest that such behavior is at least partially mediated through effects on the retinoblastoma (RB) protein, considered to be a master regulator of cell cycle, differentiation and apoptosis.

The human RB protein is a nuclear phosphoprotein spanning 928 amino acids in length that is expressed in every tissue type examined. This protein appears to be the major player in a regulatory circuit in the late G₁ (growth) phase, the so-called restriction point R, that defines a timepoint in G₁ at which cells are committed to enter S (DNA replication) phase and no longer respond to growth conditions. Moreover, RB is involved in regulating an elusive switch point between cell cycle, differentiation and apoptosis.

Functional interactions exist between RB and the three D cyclins, together with their associated kinases. Cyclins function to activate cyclin-dependent kinases, which facilitate adding phosphates onto other molecules that play a role in cell-cycle progression. The phosphorylation of RB correlates with an inactivation of its ability to arrest cellular division. Specifically, if RB is inactivated, a cell will proceed through the cell cycle, multiplying unchecked until the RB is again activated. Herein lie the implications for cancer biology. In cancer cells, the RB remains inactivated throughout its cell cycle. This results in cancer cells skipping the G₁ₚₘ phase, bypassing the restriction point R.

When the GSH concentration in NK3.3 cells is sufficiently decreased, and hence E is sufficiently increased, the RB protein in these cells cannot be phosphorylated and the cells cease to proliferate. Dephosphorylated RB traps the transcription factors that are necessary for the generation of the cyclins required for cell proliferation, resulting in a cyclin-poor cell. When GSH is restored, E is decreased, RB can be phosphorylated and these cells proliferate (Yamauchi et al., 1997). This critical value of E which induces cessation of cell proliferation (CCP), is designated E_{CCP}. Arrest in G₁ₚₘ, the first part of the G₁ phase of the cell cycle (the postmitotic interval of G₁ that lasts from mitosis (M) to the restriction point R), prevents the cell from proceeding to the second part of the G1 phase, G₁ₚₛ (the pre-S phase interval of G₁ that lasts from R to S), as well as to S and to subsequent phases of the cell cycle. When this arrest has persisted for a few hours, then the duration required for apoptosis induction is achieved. Consequently, as the cancer cells that are in G₁ₚₘ are unable to enter G₀ (Zetterberg et al., 1995), they will undergo apoptosis. In contrast, normal cells in G₁ₚₘ can, and do, enter G₀ and are able to stay there indefinitely.

Hutter et al. (1997) have studied the redox-state changes in density-dependent regulation of normal and malignant cell proliferation in the presence of modulators of GSH synthesis and have suggested a possible interrelationship between the redox potential and cell proliferation. Lee et al. (1998) showed that glucose deprivation-induced cytotoxicity is mediated by oxidative stress with formation of intracellular hydrogen peroxide in human breast carcinoma cells. Rossi et al. (1986) showed that the cytotoxicity of dimethyl- and trimethyl-benzoquinones to normal hepatocyte cells was due to a decrease in the [GSH] due to the formation of a quinone conjugate without oxidation to GSSG, while the addition of duroquinone, a tetramethylbenzoquinone, stimulated GSH oxidation and was only cytotoxic when catalase or glutathione reductase (GR) was inactivated. Smaaland et al., 1991, found a statistically significant correlation between the GSH content and the fraction of bone marrow cells in DNA synthesis.

There are many approaches for treating tumors. Some of these approaches are, to some extent, selective, such as the surgical removal of the tumor. In general, surgery is effective if the tumor has not spread and all the malignant cells have been removed. Other approaches are less selective and include radiation and chemotherapy, which usually affect normal cells as well. An agent is considered to provide a selective result if it mostly affects the cancer cells of the tumor, but does little, if any, harm to the adjacent normal cells of the tissue.

Many of the classical chemotherapeutic agents are usually more effective when the cancer cells in the tumor are rapidly proliferating. Some of the known cytotoxic agents such as vincristine, vinblastine, etoposide, methotrexate, 5-fluorouracyl, cytarabine, cisplatine, generally affect DNA during cell proliferation, primarily killing cancer cells rather than the relatively slowly proliferating normal cells. But this selectivity factor is not operative when treating slowly proliferating cancer cells. Other anti-cancer agents have been developed such tamoxifen, taxol, flavopiridol, angistatin, retinoic acid (all-trans and 9-cis), which do not affect the DNA during cell proliferation. Various mechanisms have been suggested for those two classes of agents, hereby designated as standard chemotherapeutic agents. There is, however, uncertainty in the conventional wisdom of the background art about the precise mechanisms involved. In general, anti-cancer agents, at their effective concentrations, are considered activators or triggers that trigger the formation of a sequence of various entities such as p21, which induce apoptosis (Li 1999; 2003). The concentrations of standard chemotherapeutic agents currently used for cancer treatment are limited in order to minimize injury to normal cells.

Reactive oxygen species (ROS), as generated by radiation, for example, are believed to cause mutations that produce cancer. There appears to be a consensus that antioxidants such as GSH, which can scavenge or otherwise neutralize the ROS, are required to prevent and treat cancer (Dai et al., 1999, Sen et al., 1999). If an antioxidant is defined as an agent that decreases E, by increasing the GSH²/GSSG ratio and, vice-versa, an oxidant as an agent that increases E, by decreasing the [GSH]²/[GSSG] ratio, some of the agents currently used as anticancer drugs or described in the literature as mentioned below, are clearly not acting as antioxidants.

In-vitro studies of treatment of tumor cell lines with several compounds have been carried out and have shown promising results, yet the basic mechanism of how these various compounds work remains obscure.

Dai et al. (1999) introduced As₂O₃ into various cell lines. The resulting intracellular GSH content had a decisive effect on As₂O₃-induced apoptosis, the tendency to apoptosis increased as the GSH content of the cell decreased. GSH forms an adduct with arsenic (As), viz., As(GS)₃. These researchers experimentally varied the GSH content of the various cells with BSO (buthionine sulfoximine), which inhibits gamma-glutamylcysteine synthetase, GCS, a key enzyme in GSH biosynthesis. Tendency to apoptosis increased as GSH content decreased. By itself, BSO, which caused a decrease in [GSH] of 70% in the cell, did not induce significant apoptosis, but rendered the malignant cells more sensitive to As₂O₃. The authors did not report any measured value of [GSSG]. Normal cells showed the least apoptosis.

Nicole et al. (1998) showed that the introduction of BSO to neuroblastoma cells decreased their GSH content by 98%, and induced apoptosis. Here, too, they did not report any measured value of [GSSG]. They concluded that, with these cells, there was a cause-and-effect relationship between decreasing GSH and apoptosis induction.

Sen et al. (1999) introduced α-lipoic acid into both Jurkat T-cell leukemia cells and normal lymphocytes, and noticed that the leukemia cells underwent apoptosis, whereas the normal cells did not. They suggested that the induction of apoptosis by α-lipoic acid was because this acid is a sulfur-containing antioxidant that provides strong reducing power and leads to the reduction of protein thiols.

Lizard et al. (1998) reported that the introduction of 7-ketocholesterol to U937 cancer cells induced apoptosis. They found that apoptosis was enhanced by the addition of BSO and inhibited by the addition of NAC (N-acetyl-L-cysteine), a cysteine precursor which penetrates the cell and is converted by deacetylation to cysteine, which is a GSH precursor. The authors suggested that oxidative processes are involved in 7-ketocholesterol-induced cell death.

Rudra et al. (1999) reported that the introduction of acrolein induced cytotoxicity in various cancer cell lines, such as A-427 and A-172. They demonstrated that the sensitivity to growth inhibition increases as GSH decreases. They also reported that A-427 is highly sensitive to docosahexaenoic acid, and that acrolein potentiates the cytotoxic effect of this acid. These researchers reported that acrolein depletes thiols and is highly toxic to both normal human bronchial fibroblasts and human bronchial epithelial cells in the respiratory system.

Rossi et al, (1986), Thornton et al. (1995) and Cornwell et al (1998), introduced various quinones or quinone precursors to both normal cells, such as smooth muscle cells and hepatocytes, and to leukemic cells. Rossi et al. (1986) concluded that, when GSH decreased by 90-95% of the original amount in the hepatocytes, significant cytotoxicity was induced. They all concluded that the quinones formed a Michael Adduct with the GSH.

Ramachandran et al. (1999) introduced curcumin to both human mammary epithelial cells (MCF-10A) and breast carcinoma (MCF-7/TH) cell lines, and concluded that the induction of apoptosis is due to the effect of the curcumin on some of the genes associated with cell proliferation.

Zhou et al. (1998) introduced soy isoflavones to human prostate carcinoma cells and normal vascular endothelial cells. They suggested that these soy products inhibit experimental prostate tumor growth through a combination of direct effects on tumor cells and indirect effects on tumor neovasculature.

Paschka et al. (1998) induced apoptosis of prostate cancer cell lines by introducing green tea phenols including (−)-epigallocatechin-3-gallate.

Babson and Reed (1978) describe inactivation of glutathione reductase (GR) by various isocyanates and their precursor nitrosoureas such as 2-chloroethyl isocyanate, cyclohexyl isocyanate, 1,3-bis-(2-chloroethyl)-1-nitrosourea (BCNU or carmustine), 1-(2-chloroethyl)-3-(cyclohexyl)-1-nitrosourea (CCNU or lomustine), 1-(2-chloroethyl)-3-(4-trans-methylcyclohexyl)-1-nitrosourea (MeCCNU), and 1-(2-chloroethyl)-3-(trans-4-hydroxycyclohexyl)-1-nitrosourea (trans-4-OH—CCNU). GR may also be inhibited by various antibiotics including ofloxacin, levofloxacin, cefepime, and cefazolin.

Noda et al. (2001) used colon cancer cells to test the hypothesis that cell proliferation is responsive to the cellular GSH/GSSG status. For this purpose, cells were exposed to diamide (a cell-permeant thiol agent that oxidizes GSH to GSSG) alone or in combination with BSO and/or carmustine (BCNU). The cells were either treated with BSO alone, BCNU together with diamide or all three together. BSO treatment alone markedly diminished intracellular GSH level but did not change the GSH:GSSG ratio at 30 min: this had a minimal effect on cell proliferation. On the other hand, treatment with diamide plus carmustine decreased GSH, increased GSSG, and lowered GSH:GSSG ratio within 30 min: this alteration in redox status resulted in inhibition of cell proliferation, and this effect was enhanced when cells were first pretreated with BSO. However, even with the combination of the three drugs (diamide, BSO and BCNU), cell proliferation was reduced by only approx. 50%.

Cen et al. (2002) show that disulfiram (DSF) induces apoptosis in human melanoma cells and this is a redox-related process. Melanoma cells were treated with DSF or BSO alone or in combination, and apoptosis measured. Each of the drugs induced apoptosis in the melanoma cells but, in combination, BSO only slightly enhanced the apoptosis induced by DSF. The authors suggest at the end of the paper that it will be interesting to study more drug combinations such as of DSF with cisplatin or carmustine.

Monk et al (2002) show synergistic effects of radiation therapy on human cervical carcinoma cell lines and fresh tumor explants in combination with BSO or carmustine.

Sakurai et al. (2002) shows that dimethylarsinic acid (DMA), a major human arsenic metabolite, requires intracellular GSH to induce apoptosis. Experiments were made with rat liver epithelial cell line using sodium arsenite (NaAsO₂) in combination with BSO, carmustine, diethyl maleate, or ethacrynic acid. All 4 agents enhanced the cytotoxic effects of the inorganic arsenite.

Hu et al. (2003) show a synergistic effect of BSO or ascorbic acid on arsenic trioxide in causing apoptosis of leukemia cells.

Maeda et al. (2004) show that BSO enhances in vitro growth inhibition effect of As₂O₃ on 11 different cancer cell lines and further that this combination was effective in the treatment of both primary and metastatic tumors in a mouse model of prostate cancer.

U.S. Pat. No. 6,589,987, titled “Method of Treating Cancer Using Tetraethyl Thiuram Disulfide”, shows, in Table 2, results of a cell viability experiment, in which different dosages of carmustine either alone or with a fixed amount of disulfiram was used and cell proliferation (a measurement of cell viability) was measured. The results showed a synergistic effect between carmustine and disulfiram.

With respect to tumors in general, especially slowly growing tumors, there is a dire need for agents that can selectively cause the cessation of cell proliferation (CCP), either as a result of cell arrest or apoptosis, similar to the effect of radiation on cells. Radiation is a p53 inducer, and the latter, in turn, induces p21, which can then combine with or otherwise inactivate the cyclins normally required for cell proliferation. As a result, the cyclin-poor cell undergoes cell cycle arrest or apoptosis (Gottlieb & Oren, 1996). In many cases, however, radiation is not completely selective, since it affects adjacent normal tissues; in addition, it causes unpleasant and serious side effects. Thus, more selective and effective treatments for cancer are required.

In the Israeli Patent Application No. 140970 and subsequent PCT Publication No. WO 02/056823, the applicant has disclosed a method of treating a patient with a tumor comprising administration of one or more GSH-decreasing agents. These agents either oxidize the GSH or form an adduct with GSH or inhibit GSH synthesis. Subsequent US application, published under No. US-2004-0018987, of the same applicant, discloses a method for treating a tumor in a subject comprising administering a synergistic combination of at least two agents that decrease the [GSH]²/[GSSG] ratio in the malignant cells of the tumor, wherein said agents are selected from the classes consisting of: (i) an agent that oxidizes GSH, or a precursor thereof; (ii) an agent that forms an adduct or a conjugate with GSH, or a precursor thereof; (iii) an agent that inhibits the GCS enzyme; and (iv) an agent that inhibits the glutathione reductase (GR) enzyme.

Although applicant has disclosed in the three above-mentioned applications that an agent or a combination of agents that deplete GSH can be used in a method of controlling cancer growth and inducing its regression, and synergistic combinations of two or three such agents have been proposed, none of the three applications has practical examples that show a synergistic effect of such combinations. In addition, none of the applications discloses nor teaches specific combinations of four drugs that cause and maintain the depletion of GSH, each drug acting through a different pathway, and providing effectiveness over a wide range of the concentrations of the individual agents.

Throughout this specification, various scientific publications and patents or published patent applications are referenced. Full citations for these references may be found at the end of the specification immediately preceding the claims. The disclosure of all these publications in their entireties is hereby incorporated by reference into this specification in order to more fully describe the state of the art to which this invention pertains. Citation or identification of any reference in this section or any other part of this application shall not be construed as an admission that such reference is available as prior art to the invention.

### SUMMARY OF THE INVENTION

It has now been found, in accordance with the present invention, that tumors can be effectively treated with a combination of four compounds, each compound being selected from a different category selected from categories (i) to (iv) as follows:

(i) a compound, or a precursor thereof, that oxidizes GSH;

(ii) a compound, or a precursor thereof, that forms an adduct or a conjugate with GSH;

(iii) a compound, or a precursor thereof, that inhibits the rate-limiting enzyme of GSH biosynthesis, γ-glutamylcysteine synthetase (GCS); and

(iv) a compound, or a precursor thereof, that inhibits the enzyme responsible for the conversion of GSSG to GSH, glutathione reductase (GR).

As used herein, the term “a compound or a precursor thereof” means that said compound is a metabolic product of said precursor and, thus the compound or its precursor are able to cause depletion of GSH by oxidizing GSH, forming an adduct or conjugate with GSH, inhibiting the GSC or the GR enzyme.

Thus, in one aspect, the present invention relates to a kit comprising:

(a) a container for containing a first compound (i) or a precursor thereof, said first compound or precursor being a compound that oxidizes glutathione (GSH);

(b) a container for containing a second compound (ii) or a precursor thereof, said second compound or precursor being a compound that forms an adduct or conjugate with GSH;

(c) a container for containing a third compound (iii) or a precursor thereof, said third compound or precursor being a compound that inhibits the rate-limiting enzyme of GSH biosynthesis, γ-glutamylcysteine synthetase (GCS);

(d) a container for containing a fourth compound (iv) or a precursor thereof, said fourth compound or precursor being a compound that inhibits the enzyme responsible for the conversion of GSSG to GSH, glutathione reductase (GR); and

instructions for administration of said four compounds for treatment of cancer.

A combination of compounds including at least one compound from each of the 4 categories (i) to (iv), lowers, and maintains low, the ratio of [GSH]²/[GSSG] in the malignant cells of the tumor, and thus control the redox state or environment of the malignant cells of a tumor such as to cause cessation of cell proliferation and/or apoptosis of the malignant cells.

The present invention relates, in another aspect, to a method for treatment of a cancer subject which comprises administering to said subject a pharmaceutically effective amount of at least four agents, each agent being selected from a different category selected from categories (i) to (iv) above.

In one preferred embodiment, the method of the present invention comprises administering to said subject effective amounts of said four compounds at suitable concentrations and frequency that decrease the [GSH]²/[GSSG] ratio in the malignant cells of said tumor, such as to impose on the malignant cells an E above E_{CCP}, and maintain this increased E for an appropriate duration of time that corresponds to at least the time of two to five cell cycle periods.

### BRIEF DESCRIPTION OF THE FIGURES

FIGS. 1A-1B show the effect of different combinations of disulfiram (DSF), BSO, curcumin and carmustin on the proliferation of MX-1 mammary carcinoma cells in vitro together with a negative control (no treatment) and a positive control (doxorubicin).

### DETAILED DESCRIPTION OF THE INVENTION

According to the present invention, a kit is provided comprising:

(a) a container for containing a first compound (i) or a precursor thereof, said first compound or precursor being a compound that oxidizes glutathione (GSH);

(b) a container for containing a second compound (ii) or a precursor thereof, said second compound or precursor being a compound that forms an adduct or conjugate with GSH;

(c) a container for containing a third compound (iii) or a precursor thereof, said third compound or precursor being a compound that inhibits the rate-limiting enzyme of GSH biosynthesis, γ-glutamylcysteine synthetase (GCS);

(d) a container for containing a fourth compound (iv) or a precursor thereof, said fourth compound or precursor being a compound that inhibits the enzyme responsible for the conversion of GSSG to GSH, glutathione reductase (GR); and

instructions for administration of said four compounds for treatment of cancer.

The first compound (i) or precursor thereof, such that it or its metabolic product depletes GSH by oxidizing GSH to GSSG, may be selected from the group consisting of disulfiram, diamide, diethylmalate, hydrogen peroxide precursors selected from the group consisting of ascorbic acid and dopamine, α-lipoic acid, oxidized low density lipoproteins (ox-LDLs), and a quinone selected from the group consisting of duroquinone, an ubiquinone, and β-lapachone.

The second compound (ii) or precursor thereof, such that it or its metabolic product depletes GSH by forming an adduct with GSH, may be selected from the group consisting of arsenic trioxide, ethacrynic acid, epothilones A and B, an α,β-unsaturated aldehyde or ketone, an unsubstituted or partially substituted quinone, an isoflavone, and a phenol. Preferably, said compound (ii) is selected from ethacrynic acid (EA); epothilone A and epothilone B; an α,β-unsaturated aldehyde such as cinnamaldehyde; a 4-hydroxyl-C₅-C₉-alkenal (e.g. 4-hydroxyl-pentenal, 4-hydroxyl-hexenal, 4-hydroxyl-heptenal, 4-hydroxyl-nonenal) or a precursor thereof; a polyunsaturated fatty acid (PUFA); an unsubstituted or partially substituted quinone such as anthraquinone, benzoquinone, 2-methyl-benzoquinone, 2,6-dimethyl-benzoquinone, 2,5-dimethyl-benzoquinone, and 2,3,5-trimethyl-benzoquinone, γ-tocopherolquinone and 8-tocopherolquinone; an isoflavone such as catechin, daidzein, dicumarol, (−) epicatechin, flavopiridol, genistein, β-lapachone, myricetin and rotenone; and a phenol such as curcumin, yakuchinone A, yakuchinone B, (−) epigallocatechin-3-gallate, resveratrol, γ-tocopherol, δ-tocopherol, and arsenic trioxide.

The third compound (iii) or precursor thereof, such that it or its metabolic product inhibits the rate-limiting enzyme of GSH biosynthesis, γ-glutamylcysteine synthetase (GCS), is preferably buthionine sulfoximine (BSO).

The fourth compound (iv) or precursor thereof, such that it or its metabolic product inhibits the enzyme responsible for the conversion of GSSG to GSH, glutathione reductase (GR), may be selected from various isocyanates and their precursor nitrosoureas such as 2-chloroethyl isocyanate, cyclohexyl isocyanate, 1,3-bis-(2-chloroethyl)-1-nitrosourea (BCNU or carmustine), 1-(2-chloroethyl)-3-(cyclohexyl)-1-nitrosourea (CCNU or lomustine), 1-(2-chloroethyl)-3-(4-trans-methylcyclohexyl)-1-nitrosourea (MeCCNU), and 1-(2-chloroethyl)-3-(trans-4-hydroxycyclohexyl)-1-nitrosourea (trans-4-OH—CCNU), or from various antibiotics such as ofloxacin, levofloxacin, cefepime, and cefazolin, and is preferably carmustine.

It is envisaged by the present invention that a combination of more than four agents be used for the treatment of tumors, but such combination of four or more agents should comprise at least one agent of each of the four categories (i) to (iv) in order to achieve the desired results of inhibition of tumor growth and shrinkage of existing tumors.

In the most preferred embodiment, the invention provides a kit comprising four containers in which the first compound (i) is disulfiram, the second compound (ii) is curcumin, the third compound (iii) is buthionine sulfoximine (BSO), and the fourth compound (iv) is carmustine.

The combination of the present invention treats malignancies by increasing and maintaining the intracellular redox potential above E_{CCP} that will induce selective cessation of cell proliferation and/or cell apoptosis. Although this has been proposed earlier by applicant in the above mentioned US-2001-0018987, it has now been confirmed that depletion of GSH by one agent or by a combination of two agents is not sufficient to achieve the desired results.

In the most preferred embodiment of the present invention, control of the redox state of the malignant cells refers to the control of the cellular contents of GSH and GSSG, or more particularly of the [GSH]²/[GSSG] ratio, and the combination of the invention is administered one or more times at time intervals such that the effects of the agents endure for a sufficient time in the tumor environment to decrease the [GSH]²/[GSSG] ratio in the cancer cells in the tumor and to raise the intracellular redox potential E above E_{CCP}, the redox potential where cessation of cell proliferation occurs, and to maintain this higher E continuously for an appropriate duration of time such as to induce selective apoptosis of the cancer cells. The elevated E should be maintained continuously for 2-5 cell cycles for optimum effectiveness.

Thus, according to another embodiment, the present invention provides a method for selective cessation of cell proliferation and apoptosis of malignant cells, which comprises continually administering to a subject in need pharmaceutically effective amount of four agents, each agent being selected from a different category selected from categories (i) to (iv) above, thus increasing the intracellular redox potential, E, above E_{CCP}, and maintaining this higher E for an appropriate duration of time such as to induce selective apoptosis of the cancer cells. The elevated E should be maintained for 2-5 cell cycle periods for optimum effectiveness and is within the range of from about 30 to about 250 hours, preferably from about 30 to about 200, more preferably from about 30 to about 150, still more preferably from about 30 to about 100 hours, these values depending on the type of tissue and the type and stage of the tumor.

According to the present invention, the intracellular redox potential E is expressed in millivolts (mV) and is calculated in terms of the concentrations of the members of the dominant redox couple pair GSH and GSSG according to the Nernst equation, as follows:

E=E₀−30 log [GSH]²/[GSSG],

wherein E₀ is the standard potential of glutathione.

According to the present invention, damage to the DNA is not the primary cause of cell death as with many classical chemotherapeutic agents, but rather cell death is the result of the cell undergoing apoptosis in which the DNA is damaged through the cell's own programmed death. For this reason, the agents of the combination of the invention must be administered at time intervals such that they are in contact with the cancer tissue for an appropriate time such that their effect of maintaining E above E_{CCP} is maintained continuously for the duration required to ensure that the cancer cells in the all phases of the cell cycle have had time to reach the G₁ₚₘ phase (the postmitotic interval of G₁ that lasts from M until the restriction point R), and remain in G₁ₚₘ for a sufficiently long time to permit the cell to default to apoptosis. This parameter, herein designated tau, of the administration protocol of the agent of the invention, corresponds to preferably 2-5 times the normal cell-cycle time, T. This multiple pass through the mean cell cycle period is required to allow for the variability of the cell-cycle period.

Thus, according to the present invention, the at least one agent from each of the four categories that decreases the [GSH]²/[GSSG] ratio in the malignant cells should be administered such that E remains continuously above E_{CCP} for from about 30 to about 250 hours, in order to achieve the optimum results. The time will depend on the tissue, since the cell cycle time is different from tissue to tissue, from the type of tumor and the severity of the disease. There is, however, an upper time limit for the duration of the treatment, because of the vulnerability of an organism to an E that prevents normal cells from exiting G₁ₚₘ when required; e.g. healing of wounds.

The body's homeostatic tendency to keep the concentration of bodily chemicals, in this case GSH, at a fixed value, may indicate that a one-component therapy of only removing GSH may not be effective, because the body's GSH control system will tend to replace the removed GSH. The 4-component therapy taught in the present application not only removes GSH, but attacks two important components of the GSH control system as well, in particular, one agent inactivates GCS, the rate-limiting enzyme that catalyzes the synthesis of GSH, and another agent inactivates GR, the enzyme that catalyzes the reduction of GSSG to GSH. By attacking the GSH-control system as well as by removing GSH from the cell, the present invention therapy tends to increase E and to keep it high for a significant period of time.

As shown hereinafter, the combination of the 4 agents of the present invention provides effectiveness over a wide range of the concentrations of the individual agents. This property is required if the same type tumor has spread to other parts of the body. It is also required for a “universal” therapy for different types of tumors, e.g. brain or prostate. This is because what the body takes in orally or via the blood is different from what arrives at the tumor tissue. Thus, if the agents suffer attenuation in the body by different amounts, they will arrive at the tumor tissue in both absolute and relative concentrations different from those in which they were administered. The effectiveness of the 4 agents over a wide span of concentrations offers the possibility that the concentrations that are obtained at the tumor tissue can be effective.

Thus, according to the present invention, selective induction of apoptosis of cancer cells in a tumor tissue can be obtained by imposing on this tissue, and maintaining effectively continuously, for a time, defined as tau, a well-poised redox buffer set several mV above E_{CCP}, e.g. at about −180 to −200 mV. This can be effectively achieved with the periodic administration of at least one agent from each of the four categories (i) to (iv) that decreases the [GSH]²/[GSSG] ratio, and maintains it for a time such as to achieve apoptosis. Thus, the present invention provides a method of treating a tumor in a subject, that may effectively and selectively cause the apoptosis of the malignant cells of said tumor, while constraining potential or actual harm to the normal tissues in the organism.

A “pharmaceutically effective amount” as defined herein is the amount administered at adequate frequency to maintain continuously the E of the cancer cell at about −180 to −200 mV for a time, tau, which retards the proliferation of a tumor and/or causes regression of a tumor, and constrains potential or actual harm to normal tissues in the organism.

The approach of the present invention has two types of built-in selectivity. First, normal proliferating cells may have an average E lower than the average E of cancer cells, as has been observed in fibroblasts and fibrosarcoma cells (Hutter et al., 1997). Therefore, the addition of appropriate amounts of GSH-decreasing agents to a tumor-containing tissue can increase the E of the cancer cells to or beyond E_{CCP}, whereas the E of normal proliferating cells in the tissue can still remain below E_{CCP} (Hoffman et al., 2001). Second, normal cells that are trapped in G₁ₚₘ enter G₀ where they may remain indefinitely. Cancer cells, on the other hand, cannot enter G₀. Instead, after several hours in G₁ₚₘ, they undergo apoptosis. Consequently, the introduction of agents according to the invention will cause apoptosis in cancer cells and will not harm normal cells.

The term “tumor” as used herein encompasses all types of malignant cells, cancerous cells, cancers and malignant tumors. It includes non-solid tumors such as leukemias and lymphomas, and solid tumors including, but not being limited to, bladder, bone, brain, breast, cervical, colon, esophageal, kidney, laryngeal, liver, lung, melanoma, ovary, pancreas, prostate, rectal, skin, testicular, and uterine tumors. Moreover, the term “tumor” encompasses primary tumors, secondary tumors, and metastases thereof in the same organ or in another organ. It is envisaged that this invention will work preferably in tumor cells in which the RB protein is operative. If, however, elevated E stops proliferation more by inactivating the transcription factors than by preventing phosphorylation of pRB, then the invention will work even if pRB is not operative.

The terms “treatment of a tumor” and “anti-tumor” as used herein refer to a treatment or a composition that retards the proliferation of a tumor and/or causes regression of a tumor.

In one embodiment of the invention, the at least four [GSH]²/[GSSG]-decreasing agents may be administered together with at least one standard chemotherapeutic drug such as, but not limited to, vincristine, vinblastine, melphalan, methotrexate, 5-fluorouracyl, cytarabine, cisplatine, tamoxifen, taxol, angistatin, and/or in conjunction with a non-drug treatment for cancer such as radiotherapy.

Besides the agents mentioned above, it should be mentioned that certain conditions might also decrease the cellular [GSH]²/[GSSG] ratio. For example, radiation therapy, glucose deprivation (Lee et al., 1998), hyperthermia (Lord-Fontaine and Averill, 1999) and hypoxia (Araya et al, 1998), and methods employing the above agents and these conditions as complementary therapy are envisaged by the present invention.

Based on the differences in kinetics and mechanisms of the various types of agents (i) to (iv), the combinations of the invention will provide varying degrees of synergy with respect to not only increasing, but also maintaining this increase in the [GSH]²/[GSSG] ratio, so that the in vivo frequency of administration is no more than 2-3 times a day.

Many of the standard chemotherapeutic agents are conventionally considered to be antioxidants. If they act as reducing agents that increase GSH, decrease GGSG and decrease E, they will permit the RB protein to remain or become phosphorylated, allowing cell proliferation. Thus, whereas antioxidants might prevent cancer, e.g. by scavenging/neutralizing reactive oxygen species, they will enhance the proliferation of cells that have already become cancerous. Without being limited to a single hypothesis, the novel approach of the present invention applies the anti-proliferative effect of the dephosphorylated (hypophosphorylated) RB protein to halt the progress of the cell through its cycle by increasing E. This will be applicable for any cancer having an operational RB protein (pRB). And if the effect of redox is primarily on the transcription factors rather than on the pRB, the method should work even if pRB is not functional.

A preferred feature of the present invention is to “match” the [GSH]²/[GSSG]-decreasing agents to the location of the specific non-metastasizing tumor. For example, when the method of the invention is applied to a patient with a brain tumor, the [GSH]²/[GSSG]-decreasing agents should preferably be relatively small molecules in order to optimize their passage through the blood-brain barrier, e.g. dopamine as hydrogen peroxide precursor.

The agents of the invention in the separate containers may be in the form of a pharmaceutical composition for direct administration, for example as oral formulations or formulations for parenteral administration, with a suitable pharmaceutically acceptable carrier and possibly other excipients. As used herein, the term “pharmaceutically acceptable carrier” encompasses any of the standard pharmaceutical carriers. Such carriers are well known in the art and may include, but are in no way and are not intended to be limited to, any of the standard pharmaceutical carriers such as phosphate-buffered saline (PBS) solutions, water, emulsions such as oil/water emulsion, suspensions, and various types of wetting agents. Typically, such carriers contain excipients such as starch, milk, sugar, certain types of clay, gelatin, stearic acid or salts thereof, magnesium or calcium stearate, talc, vegetable fats or oils, gums, glycols, or other known excipients. Such carriers may also include flavor and color additives, preservatives and the like, as well as other ingredients.

The pharmaceutical compositions for use according to the invention are formulated by well-known conventional methods. The compositions of this invention may include sterile solutions, tablets, coated tablets, capsules, pills, ointments, creams, lotions, gels, suppositories, pessaries, drops, liquids, sprays, powders, patches or any other means known in the art.

The agents may be administered by any of the well-known and suitable methods of administration, including, but not limited to, intravenous, intramuscular, intravesical, intraperitoneal, topical, transdermal (for example, using a patch containing one or more agents according to the invention), transmucosal, subcutaneous, rectal, vaginal, ophthalmic, pulmonary (inhalation), nasal, oral and buccal administration, by inhalation or insufflation (via the nose or mouth), administration as a coating to a medical device (e.g. a stent) and slow-release formulations (or packaging).

The instructions provided in the kit of the present invention should describe the protocol of administration of the four agents that achieves the goal of the present invention. For example, one or more of the drugs may be administered orally and one or more may be administered by injection.

The dosage to be administered will vary according to the type of tumor, the severity of the disease and the age and condition of the patient and will be determined by the physician skilled in the art. In a preferred embodiment, the agents are administered 1-4 times a day, such that the total amount per day should be from 0.01 g to 150 g, during as many days as to ensure the effective presence of the agent or agents, i.e. the maintenance of E above E_{CCP}, for a time, tau.

The four agents of the invention may be administered simultaneously, or preferably, subsequently to each other. For example, for the combination of disulfiram, BSO, curcumin and carmustine, it may be preferable to first administer BSO and carmustine intravenously, and subsequently disulfiram and curcumin per os.

In one embodiment, the present invention relates to a method of selectively inducing apoptosis in cancerous cells which comprises administering to a subject an effective amount of a combination of 4 compounds that increases the redox potential of the cancerous cells to a threshold potential that induces apoptosis while the increase in redox potential in non-cancerous cells does not induce apoptosis, wherein in said combination of 4 compounds each compound belongs to a different category selected from the group consisting of categories (i) to (iv). In a preferred embodiment, the redox potential of the cancerous cells is increased and maintained in the range of about −200 to −180 mV.

In another embodiment, the present invention relates to a method of selectively inducing apoptosis in cancerous cells which comprises administering to a subject an effective amount of a combination of 4 compounds that prevents phosphorylation of the retinoblastoma protein (pRB) in said cancerous cells, wherein in said combination of 4 compounds each compound belongs to a different category selected from the group consisting of categories (i) to (iv).

In a further embodiment, the present invention relates to a method of selectively inducing apoptosis in cancerous cells which comprises administering to a subject an effective amount of a combination of 4 compounds that prevents release of transcriptional factors from the retinoblastoma protein (pRB) in said cancerous cells, wherein in said combination of 4 compounds each compound belongs to a different category selected from the group consisting of categories (i) to (iv).

In still another embodiment, the present invention relates to a method of selectively inducing apoptosis in cancerous cells which comprises administering to an individual in need an effective amount of a combination of 4 compounds such that the four compounds remain in contact with the cancer cells for the duration of time required to ensure that the cancer cells do not enter G₀ and therefore apoptosis is induced when they remain in G₁ₚₘ, wherein each compound of the combination of 4 compounds belongs to a different category selected from the group consisting of categories (i) to (iv). Preferably, said duration of time corresponds to 2-5 times the normal cell-cycle time, and is from about 30 to about 250 hours.

In yet another embodiment, the present invention relates to a method of selectively inducing apoptosis in cancerous cells by decreasing the [GSH]²/[GSSG] ratio such that the redox potential E in cancerous cells is increased to or above the threshold potential while the increase in redox potential in non-cancerous cells is such that it remains below the threshold potential, which comprises administering to an individual in need an effective amount of a combination of 4 compounds wherein each compound belongs to a different category selected from the group consisting of categories (i) to (iv).

In yet a further embodiment, the present invention relates to a method of selectively inducing apoptosis in cancerous cells in a subject comprising administering to said subject a therapeutically effective amount of a combination of 4 compounds wherein each compound belongs to a different category selected from the group consisting of categories (i) to (iv).

In yet a further embodiment, the present invention relates to a method of inducing apoptosis in cancerous cells or tissues comprising contacting cancerous cells or tissues with a combination of 4 compounds wherein each compound belongs to a different category selected from the group consisting of categories (i) to (iv). In a preferred embodiment, the combination of the 4 compounds is administered to a subject in vivo in an amount sufficient to induce apoptosis of cancerous cells or tissue in said subject either by altering the intracellular redox potential such that the redox potential in cancerous cells is increased to a threshold potential sufficient to prevent phosphorylation of the retinoblastoma protein (pRB) while the increase in redox potential in non-cancerous cells is insufficient to prevent phosphorylation of the retinoblastoma protein in said cells, or by altering the intracellular redox potential such that the redox potential in cancerous cells is increased to a threshold potential sufficient to prevent release of transcriptional factors from the retinoblastoma protein (pRB) in said cancerous cells.

The invention further relates to a method of treating cancer by altering the intracellular redox potential such that the redox potential in cancerous cells is increased to the threshold potential to induce apoptosis, by treating the cancer cells with an effective amount of a combination of 4 compounds wherein each compound belongs to a different category selected from the group consisting of categories (i) to (iv), to decrease and maintain a low [GSH]²/[GSSG] ratio. In a preferred embodiment, the cancer cells are in a mammal.

The invention still further relates to a method of inducing apoptosis in a tumor cell comprising the step of administering to a cell culture or mammalian host having said tumor cell an effective amount of a combination of 4 compounds wherein each compound belongs to a different category selected from the group consisting of categories (i) to (iv).

The invention further relates to a method for treatment of cancer comprising the step of administering to a patient in need an effective amount of a combination of 4 compounds wherein each compound belongs to a different category selected from the group consisting of categories (i) to (iv).

In another aspect, the invention relates to the use of a combination of 4 compounds wherein each compound belongs to a different category selected from the group consisting of categories (i) to (iv), for the manufacture of a kit for treatment of cancer.

The invention will be illustrated by the following illustrative and non-limitative Examples.

### EXAMPLES

### Example 1

### Effect of a Combination of Disulfiram, BSO, Curcumin and Carmustine on Proliferation of MX-1 Mammary Carcinoma Cells

### Material and Methods

Test compounds: Tested substances were dissolved in the appropriate medium as follows: carmustine (Sigma) was dissolved in ethanol, disulfiram (DSF) (Sigma) and curcumin (Fluka) were dissolved in dimethyl sulfoxide (DMSO), and DL-buthionine-sulfoximine (BSO) (Fluka) was dissolved in growth medium.

Compounds were added in a 1:10 serial dilution to the wells to obtain range of 10⁻³ M to 10⁻⁸ M final concentrations.

Controls: Positive control—10⁻⁵M Doxorubicin. Negative control—Media containing the compound's solvent.

Media. buffers, solvents: washing buffer: PBS at 4° C.; 5% trichloroacrtic acid (TCA) solution; 10.25M NaOH; and microscintilation liquid (Microscint 20™)

Cell lines: MX-1 mammary carcinoma cells were grown in RPMI-1640 medium, supplemented with 10% FBS, 2 mM L-glutamine, 1% penicillin/streptomycin and 1% non-essential amino acids.

MX-1 culturing and sub-culturing: MX-1 cells were grown in 75 cm² culture flasks. The culture medium was changed every other day and the day before the experiment. For sub-culturing, the medium was removed and the cells were detached from the culture flasks with 0.25% trypsin-ethylenediaminetetraacetic acid (EDTA). Culture medium with fetal bovine serum was added to stop trypsinization. The cultures were kept at 37° C. in an atmosphere of 5% CO₂ and 100% humidity. Cells were diluted to a density of 2500 cells/well in 96-well microscintilation plates (Packard). Cells were in the logarithmic phase for the whole time of the experiment.

Compounds administration: One day after seeding the cells, compounds were added to the plates to a final concentration of 10⁻³M to 10⁻⁸M.

Thymidine incorporation (proliferation) assay and cells harvesting: 24 hours after administering the compounds, to each well in the culture plates, ³H-thymidine (NET-027 Thymidine [methyl-³H] from NEN, 6.7 Ci/mmol) was added to final concentration of 0.4 μCi/well. Plates were returned to the incubator for another 48 hours and then harvested. Medium was collected and cells were washed twice with PBS at 4° C. Cells were incubated with 5% TCA at 4° C. for 20 minutes. Lysis was done by adding 10.25M NaOH for 30 minutes with shaking. Radioactivity was determined after adding 180 μl scintillation liquid (Microscint 20™, Packard).

Data calculation: The data represent the mean amount of thymidine counted per well (CPM). Average and standard deviation (SD) were calculated for each plate (12 wells). In order to compare between compounds, the average of negative control (medium with solvent) is presented as 100% and all other tested concentrations are translated respectively. Statistical analysis of raw data was conducted using InStat software. One-way ANOVA was performed with multiple comparisons post-test according to Dunnett for treatments vs. control, and according to Tukey for treatments comparisons.

### Results:

In order to examine whether the combination of the four compounds, disulfiram, carmustin, BSO and curcumin, has synergistic effects, MX-1 cells were treated with different combinations and concentrations of the 4 compounds, summarized in Table 1:

TABLE 1
Range of concentrations of the four agents

| Category | Agent | Range of Concentrations |
|---|---|---|
| 1 | Disulfiram | 10⁻⁴-10⁻⁷ M |
| 2 | Curcumin | 10⁻⁴-10⁻⁷ M |
| 3 | BSO | 5 × 10⁻³-10⁻⁵ M |
| 4 | Carmustine | 10⁻⁶-2 × 10⁻⁷ M |

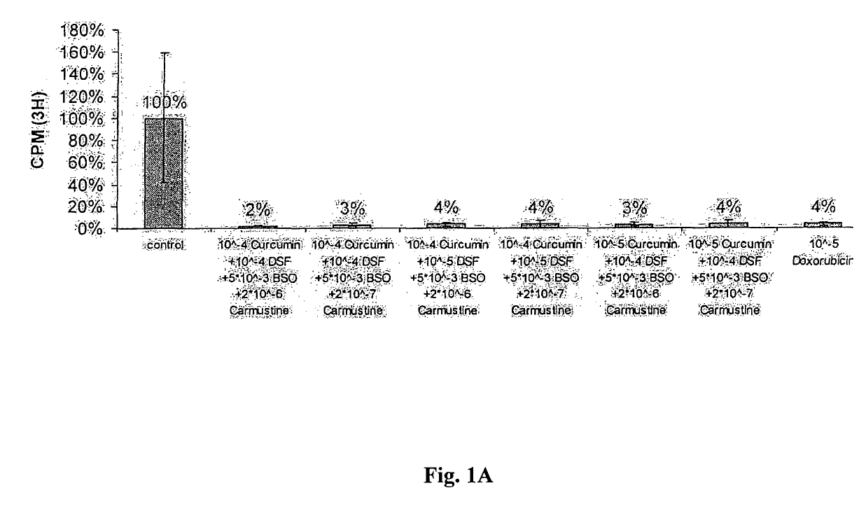

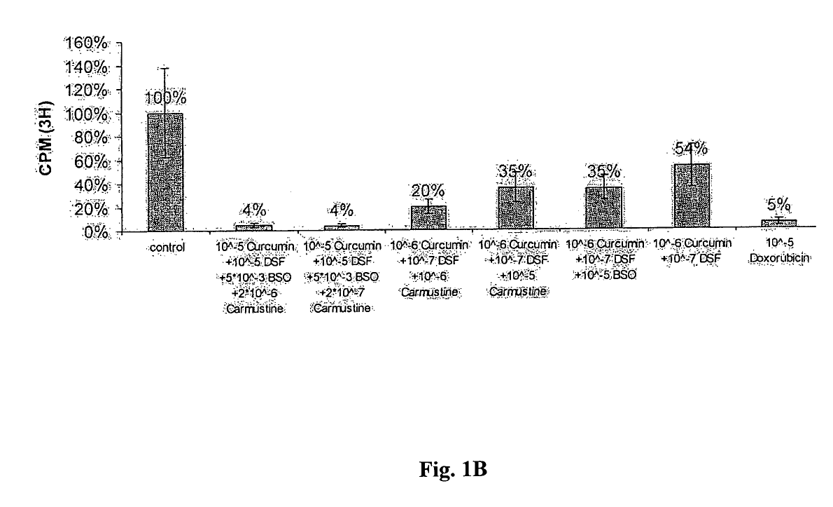

Cell proliferation was determined by ³H-Thymidine assays and the results are depicted in FIGS. 1A-1B and summarized in Table 2.

TABLE 2
Effect of DSF, BSO, curcumin and
carmustine on MX-1 proliferation

| Disulfiram | Curcumin | BSO | Carmustine | Cell Proliferation |
|---|---|---|---|---|
| Conc (M) | Conc (M) | Conc (M) | Conc (M) | (%) |
| 0 | 0 | 0 | 0 | 100 |
| 10⁻⁴ | 10⁻⁴ | 5 × 10⁻³ | 2 × 10⁻⁶ | 2 |
| 10⁻⁴ | 10⁻⁴ | 5 × 10⁻³ | 2 × 10⁻⁷ | 3 |
| 10⁻⁵ | 10⁻⁴ | 5 × 10⁻³ | 2 × 10⁻⁶ | 4 |
| 10⁻⁵ | 10⁻⁴ | 5 × 10⁻³ | 2 × 10⁻⁷ | 4 |
| 10⁻⁴ | 10⁻⁵ | 5 × 10⁻³ | 2 × 10⁻⁶ | 3 |
| 10⁻⁴ | 10⁻⁵ | 5 × 10⁻³ | 2 × 10⁻⁷ | 4 |
| 10⁻⁵ | 10⁻⁵ | 5 × 10⁻³ | 2 × 10⁻⁶ | 4 |
| 10⁻⁵ | 10⁻⁵ | 5 × 10⁻³ | 2 × 10⁻⁷ | 4 |
| 10⁻⁷ | 10⁻⁶ | | 10⁻⁶ | 20 |
| 10⁻⁷ | 10⁻⁶ | | 10⁻⁵ | 35 |
| 10⁻⁷ | 10⁻⁷ | 10⁻⁵ | | 35 |
| 10⁻⁷ | 10⁻⁶ | | | 54 |

Cell proliferation rate with the positive control, 10⁻⁵ M doxorubicin, was 4-11%.

Different concentrations of two agents (DSF and carmustine) or three agents (DSF, carmustine and BSO or DSF, carmustine and curcumin or DSF, BSO, and curcumin) were tested on MX-1 cell proliferation. Several of these combinations showed reduced cytotoxicity and were significantly less active (around 30-50% survival). These concentrations are shown in Table 3.

TABLE 3
Combinations of 2 or 3 agents with reduced cytotoxicity

| | | | | Significance |
|---|---|---|---|---|
| Disulfiram | Carmutine | BSO | Curcumin | when compared to |
| Conc (M) | Conc (M) | Conc (M) | Conc (M) | all treatments |
| 2 × 10⁻⁷ | 2 × 10⁻⁷ | 10⁻⁴ | 0 | p < 0.001 |
| 2 × 10⁻⁵ | 2 × 10⁻⁶ | 10⁻³ | 0 | p < 0.001 |
| 2 × 10⁻⁵ | 2 × 10⁻⁶ | 0 | 0 | p < 0.001 |
| 10⁻⁷ | 10⁻⁶ | 0 | 10⁻⁶ | p < 0.001 |
| 10⁻⁷ | 10⁻⁵ | 0 | 10⁻⁶ | p < 0.001 |
| 10⁻⁷ | 0 | 10⁻⁵ | 10⁻⁶ | p < 0.001 |
| 10⁻⁷ | 0 | 0 | 10⁻⁶ | p < 0.001 |

As shown in FIGS. 1A and 1B, the four-drug combination comprising different concentrations of the four agents DSF, BSO, curcumin and carmustine was as effective or more effective in reducing cell proliferation (2-4%) than the known chemotherapeutic agent doxorubicin 4%. In addition, Table 3 above demonstrates that the effectiveness of the combinations comprising 2 or 3 agents are affected by the concentration of the individual components used, whereas FIG. 1 shows that the 4-agent combinations remained effective over all 8 of the concentrations that were tested. These results show that the combination of the 4 agents is less sensitive to concentration effect than the combination of 2 or 3 agents. These results may be explained by the enhanced duration of the higher E, even if contact between cells and the agents in the wells is interrupted.

### Example 2

### Antitumor Activity of the 4-Drug Combination in CD-1-nu Mice

Following the positive synergistic results obtained in vitro, the safety and effect of the combination of DSF, BSO, curcumin and carmustine was then tested in CD-1 nude (nu/nu) mice implanted with the MX-1 human breast carcinoma cells.

### Materials and Methods

Test compounds. Carmustine (Sigma) dry powder was dissolved initially in ethanol to a concentration of 100 mg/ml, and further diluted in saline to obtain a solution of 13.3 mg/ml. Curcumin (Fluka) dry powder was dissolved initially in absolute ethanol and further diluted in water to obtain a solution of 25 mg/ml, containing not more than 10% ethanol of the final volume. BSO (Fluka) dry powder was dissolved in water to obtain a solution of 50 mg/ml. DSF (minimum 98.0%) (Sigma) as solid granular particles was dissolved initially in DMSO and further diluted in water to obtain a solution of 2 mg/ml, containing not more than 5% DMSO of the final volume.

Test System: Female CD-1 nu mice (n=21) were used in this experiment. The mice were 6-8 weeks old at the onset of the study. The mice were divided into three groups: 1F—untreated controls (tumor-bearing mice, untreated); 2F—tumor-bearing mice treated with the four compounds; 3F—non-tumor bearing mice treated with the four compounds (safety control). Table 4 summarizes the constitution of the test groups and dose levels.

TABLE 4
Constitution of mice test groups and dose levels.

| | Tumor | | | | | |
|---|---|---|---|---|---|---|
| Gp. | induction | No. of | | Doses | Administration | |
| No. | (+/−) | Animals | Treatment | (mg/kg) | Route | Frequency |
| 1F | + | 8 | Untreated | 0.0 | NA | NA |
| | | | Control | | | |
| 2F | + | 8 | Curcumin | 250 | PO | Day 1 to |
| | | | Carmustine | 66.3 | IV | Day 3 |
| | | | BSO | 500 | IV | |
| | | | DSF | 20 | PO | |
| 3F | − | 5 | Curcumin | 250 | PO | Day 1 to |
| | | | Carmustine | 66.3 | IV | Day 3 |
| | | | BSO | 500 | IV | |
| | | | DSF | 20 | PO | |

NA = Not applicable; IV = Intravenous; PO = Per os (oral)

Dose level selection: Doses for carmustine and BSO were specified in mg or g per m², respectively, and were interpreted for mg/kg according to online website Dose-Calculator (www.fda.gov/cder/cancer/animalquery.htm). The dose for BSO represents the maximal feasible dose considering the solubility of the test compound and the maximal volume dosage that can be administered.

Administration: In view of the large volume that had to be administered to animals on the same day (i.e. twice PO and twice IV administrations), the animal received the compounds in two phases. Initially, the compounds administered by IV, carmustine and BSO, were given, and 30 minutes later the other two compounds administered PO, curcumin and DSF, were given. In all instances, the compounds were administered at a constant volume not to exceed 5 ml/kg for IV administration or 10 ml/kg for PO administration.

Tumor Induction: The tumorogenic substance (Mammary Xenograft-1, MX-1) is a human derived mammary duct carcinoma, supplied by the National Cancer Institute (NCI). The total tumor mass (16 fragments, one per each transplanted animal in the study) was prepared according to the NCI recommended transplantation protocol as described below.

Tumor Propagation: Tumor-bearing animals serving as donors were euthanized. The tumor was excised, dissected and transferred to a sterile petri dish placed on ice. The tumor mass was cut into approximately 30 fragments (2×2×2 mm each) and transplanted into 30 naive anesthetized mice in the subcutaneous flank region.

Tumor Monitoring (Pre-Treatment): Tumor growth monitoring was performed at least twice weekly. Measurements were done using a Mitutoyo Electronic Digital Caliper. All the tumor-bearing mice had a tumor volume ranging from 150-200 mm³ at the onset of treatment.

Observations and Examinations: Tumor-bearing groups (1F and 2F) were observed for seven days following the last treatment, unless signs of remission were evident, at which case the observation period was extended to 14 days. The safety group (3F) was observed for 42 days following last treatment.

Examinations: For clinical signs, the mice were frequently examined on the day they received the doses and thereupon at least once daily on working days. Body weight was measured just prior to treatment on Days 1, 2, 3 and thereafter once weekly until study termination. Tumor size measurements (volume) were carried out on Day 1, Day 2 and thereafter every other day until study termination. Calculation of the tumor volume was done according to the following equation:

V(mm³)=d²(mm²)×D(mm)/2

where d and D represent the smallest and the largest perpendicular tumor diameters, respectively. Ulcerated tumors were not measured. Only the last measured value prior to ulceration was considered for data evaluation.

Results:

There was no incidence of mortality after 45 days in Group 3F (non-bearing tumor mice that received treatment with the 4 agents in three successive days). In addition, the 5 mice of this group gained weight, as shown in Table 5. This indicates that the combination of the 4 compounds at the dosages used is safe.

TABLE 5
Mean group (±SD) body weight and gain (g) values in CD-1-nu female mice following three
repeated administrations on 3 successive days of carmustine and BSO by the intravenous
(IV) route, followed by administration of curcumin and DSF by the oral (PO) route

| | | Body Weight (g) - Day No. | | | | | | | | Gain (Day 45 |
|---|---|---|---|---|---|---|---|---|---|---|
| Group | | 1 | 3 | 10 | 18 | 25 | 32 | 38 | 45 | vs. Day 1) |
| 3F | Mean ± SD | 20.1 | 19.6 | 21.4 | 21.9 | 22.2 | 22.2 | 23.6 | 23.9 | 3.8 |
| (n = 5) | | 2.62 | 3.17 | 2.92 | 3.09 | 2.96 | 2.62 | 2.98 | 3.21 | 0.85 |

Measurements of tumor volume were made in the 8 animals of each of the 2 sets: Group 1F (tumor-bearing mice, untreated) and Group 2F (tumor-bearing mice, treated with combination of DSF, BSO, curcumin and carmustine). The group 2F animals were dosed on 3 consecutive days and the tumor volumes were measured on Days 1, 2, 4, 6 and 8, where day 1 is the day of the initial dosing. Table 6a lists the tumor volumes of each of the animals on day 1 and day 2 and the difference between them.

TABLE 6a
Tumor volume data for Day 1 and Day
2, untreated and treated animals

Tumor Volume

| | Group | Animal No. | 1 | 2 | Vol dif |
|---|---|---|---|---|---|
| | 1F | 17 | 134 | 124 | −10.0 |
| | Untreated | 27 | 159 | 217 | 58.2 |
| | | 28 | 191 | 213 | 21.2 |
| | | 21 | 209 | 266 | 57.0 |
| | | 20 | 131 | 122 | −9.0 |
| | | 15 | 159 | 204 | 44.4 |
| | | 45 | 179 | 200 | 21.0 |
| | | 26 | 129 | 182 | 52.4 |
| Mean | | | 162 | 191 | 29.4 |
| SD | | | 29.5 | 48.2 | 28.1 |
| | 2F | 35 | 136 | 169 | 33.0 |
| | Treated | 48 | 154 | 209 | 54.6 |
| | | 50 | 165 | 220 | 55.6 |
| | | 40 | 193 | 271 | 77.1 |
| | | 42 | 193 | 118 | −75.2 |
| | | 11 | 148 | 112 | −36.6 |
| | | 47 | 144 | 173 | 29.2 |
| | | 46 | 169 | 131 | −37.7 |

| | | | |
|---|---|---|---|
| Mean | 161 | 186 | 12.5 |
| SD | 21.6 | 53.8 | 54.9 |

The treatments to Group 2F (treated) were not initiated on the same day, but rather, over several days, in groups of 1, 2, and 3 mice each, in the order listed. Animals 42, 11, 47, and 46 were dosed later than the other 4 mice and they are designated, respectively, as the later-dosed and early-dosed mice. In this experiment, a non-conventional dosage protocol was employed (See Dosage Protocol) and perhaps for this reason the data indicate that there was a dosing problem. The dosing seems to have been poor in the beginning and to have improved with time. This improvement is supported by the correlation of the data with dosage experience as described below.

The eight treated mice appear to divide into two distinct groups based on the growth or regression of the tumors. The first 4, that are the early-dosed animals, show a large mean-tumor-volume increase from Day 1 to Day 2, whereas the later-dosed 4 show a mean-tumor-volume decrease. A statistical significance test of the mean-tumor-volume change from Day 1 to Day 2, shows that the two groups are different at the level of p=0.01. Because the dosing appears to be better for the later-dosed 4 than for the early-dosed, the data on the 4 later-dosed animals are taken as more representative of the efficacy of the therapy than the data of the 4 early-dosed animals.

TABLE 6b
Tumor-volume differences in Days 1-2 and
Days 1-4 for the 4 later-dosed animals.

| Animal # | Day 1-2 | Day 1-4 |
|---|---|---|
| 42 | −75 | 28 |
| 11 | −37 | 21 |
| 47 | 29 | 220 |
| 46 | −38 | −62 |
| Mean | −30 | 52 |
| Std Dev | 43 | 119 |

Table 6b shows the difference in volume measurements in mm³ between Day 1 and Day 2, and between Day 1 and Day 4. The mean volume difference of the group from Day 1 to Day 2 showed regression. The mean volume difference of the group from Day 1 to Day 4 showed growth, but that could be mainly attributed to animal #47, which seems to have been subject to inadequate dosing.

Table 6a shows that the untreated tumors are growing normally as expected from Day 1 to Day 2. The treatment is intended to slow this growth and even reverse it. As shown for the later-dosed subset of treated mice, the treatment is having an effect because the mean value of the differences for the treated tumors is negative.

However, even if all eight treated mice are considered as a group, an effect can be observed. In contrast to the untreated group of 8 animals, which demonstrate a statistically significant (p=0.01) increase in tumor volume (ΔV=29.4) from Day 1 to Day 2, there was no significant (p=0.27) difference in mean-tumor-volume (ΔV=12.5), between Day 1 and Day 2, in the treated group of 8 animals. This demonstrates that there was a definite slowing of tumor growth of the 8 treated animals as a group, compared to the 8 untreated animals.

### REFERENCES

Araya R, Uehara T, Nomura Y. (1998) “Hypoxia induces apoptosis in human neuroblastoma SK—N-MC cells by caspase activation accompanying cytochrome c release from mitochondria.” FEBS Lett 439:168-72
Cen D., Gonzalez R. I., Buckmeier J. A., Kahlon R. S., Tohidian N. B., Meyskens F. L. Jr. (2002) “Disulfiram induces apoptosis in human melanoma cells: a redox-related process.” Molec Cancer Ther 1: 197-204.
Cornwell D. G., Jones K. H., Jiang Z., Lantry L. E., Southwell-Keely P., Kohar I., Thornton D E (1998) “Cytotoxicity of tocopherols and their quinones in drug-sensitive and multidrug-resistant leukemia cells.” Lipids 33:295-301.
Dai J., Weinberg R. S., Waxman S., Jing Y. (1999) “Malignant cells can be sensitized to undergo growth inhibition and apoptosis by arsenic trioxide through modulation of the glutathione redox system.” Blood 93: 268-77.
Gottlieb T M, Oren M. (1996) “p53 in growth control and neoplasia.” Biochim Biophys Acta 1287: 77-102.
Hoffman A., Spetner L. M., Burke M. (2001) “Cessation of cell proliferation by adjustment of cell redox potential.” J Theoret Biol 211:403-7.
Hu X. M., Hirano T, Oka K. (2003) “Arsenic trioxide induces apoptosis in cells of MOLT-4 and its daunorubicin-resistant cell line via depletion of intracellular glutathione, disruption of mitochondrial membrane potential and activation of caspase-3.” Cancer Chemotherapy & Pharmacology 52: 47-58.
Hutter D. E., Till B. G., Greene J. J. (1997) “Redox state changes in density-dependent regulation of proliferation.” Exp Cell Res 232:435-438.
Lee Y J, Galoforo S. S., Berns C. M., Chen J. C., Davis B. H., Sim J. E., Corry P. M., Spitz D. R. (1998) “Glucose deprivation-induced cytotoxicity and alterations in mitogen-activated protein kinase activation are mediated by oxidative stress in multidrug-resistant human breast carcinoma cells”. J Biol Chem 273: 5294-9.
Li Y., Sun X., LaMont J. T., Pardee A. B., Li C. J. (2003) “Selective killing of cancer cells by beta-lapachone: direct checkpoint activation as a strategy against cancer.” Proc Natl Acad Sci USA 100:2674-8.
Li C. J., Li Y. Z., Pinto A. V., Pardee A. B. (1999) “Potent inhibition of tumor survival in vivo by beta-lapachone plus taxol: combining drugs imposes different artificial checkpoints.” Proc Natl Acad Sci USA. 96:13369-74.
Lizard G., Gueldry S., Sordet O., Monier S., Athias A., Miguet C., Bessede G., Lemaire S., Solary E., Gambert P. (1998) “Glutathione is implied in the control of 7-ketocholesterol-induced apoptosis, which is associated with radical oxygen species production.” FASEB J 12: 1651
Lord-Fontaine S., Averill D. A. (1999) “Enhancement of cytotoxicity of hydrogen peroxide by hyperthermia in chinese hamster ovary cells: role of antioxidant defenses.” Arch Biochem Biophys 363:283-95.
Maeda H., Hori S., Ohizumi H., Segawa T., Kakehi Y., Ogawa O., Kakizuka A. (2004) “Effective treatment of advanced solid tumors by the combination of arsenic trioxide and L-buthionine-sulfoximine.” Cell Death Difer. 2004 Mar. 5 [E-published ahead of print]
Monk B. J., Burger R. A., Parker R., Radany E. H., Redpath L., Fruehauf J. P. (2002) “Development of an in vitro chemo-radiation response assay for cervical carcinoma.” Gynecol. Oncol. 87:193-99
Nicole A., Santiard-Baron D., Ceballos-Picot I. (1998) “Direct evidence for glutathione as mediator of apoptosis in neuronal cells.” Biomed Pharmacother 52: 349-55.
Noda T., Iwakiri R., Fujimoto k., Aw T. Y. (2001) “Induction of mild intracellular redox imbalance inhibits proliferation of CaC0-2 cells” FASEB J 15: 2131-2139.
Paschka A. G., Butler R., Young C. Y. (1998) “Induction of apoptosis in prostate cancer cell lines by the green tea component, (−)-epigallocatechin-3-gallate.” Cancer Lett 130:1-7.
Ramachandran C., You W. (1999) “Differential sensitivity of human mammary epithelial and breast carcinoma cell lines to curcumin.” Breast Cancer Res Treat 54:269-78.
Rossi L., Moore G. A., Orrenius S., O'Brien P. J. (1986) “Quinone toxicity in hepatocytes without oxidative stress.” Arch Biochem Biophys 251: 25-35.
Rudra P. K., Krokan H. E. (1999) “Acrolein cytotoxicity and glutathione depletion in n-3 fatty acid sensitive- and resistant human tumor cells.” Anticancer Res 19: 461-9.
Sakurai T., Qu W., Sakurai M H, Waalkes M. P. (2002) “A major human arsenic metabolite, dimethylarsinic acid, requires reduced glutathione to induce apoptosis.” Chem. Res. Toxicology 15: 629-37
Sen C. K., Sashwati R., Packer L. (1999) “Fas mediated apoptosis of human Jurkat T-cells: intracellular events and potentiation by redox-active alpha-lipoic acid.” Cell Death Differentiation 6:481-91.
Smaaland R., Svardal A. M., Lote K., Ueland M., Laerum O. D. (1991) “Glutathione content in human bone marrow and circadian stage relation to DNA synthesis.” J Natl Cancer Inst 83:1092-8.
Thornton D. E., Jones K. H., Jiang Z., Zhang H., Liu G., Cornwell D. G. (1995) “Antioxidant and cytotoxic tocopheryl quinones in normal and cancer cells.” Free Radic Biol Med 18:963-76.
Yamauchi A., Bloom E. T. (1997) “Control of cell cycle progression in human natural killer cells through redox regulation of expression and phosphorylation of retinoblastoma gene product protein” Blood 89: 4092-409.
Zetterberg A. and Larsson O. (1995) “Cell cycle progression and cell growth in mammalian cells”, in Frontiers in Molecular Biology: Cell Cycle Control, Ed. by Hutchings C and Glover D M, p 2066, Oxford University Press, Oxford, UK.
Zetterberg A., Larsson O., Wiman K. G. (1995) “What is the restriction point?” Curr Opinion in Cell Biology 7:835-42.
Zhou J. R., Gugger E. T., Tanaka T., Guo Y., Blackburn G. L., Clinton S. K. (1999) “Soybean phytochemicals inhibit the growth of transplantable human prostate carcinoma and tumor angiogenesis in mice.” J Nutrition 129:1628-35.

1. A kit comprising:
(a) a container for containing a first compound (i) or a precursor thereof, said first compound or precursor being a compound that oxidizes glutathione (GSH);
(b) a container for containing a second compound (ii) or a precursor thereof, said second compound or precursor being a compound that forms an adduct or conjugate with GSH;
(c) a container for containing a third compound (iii) or a precursor thereof, said third compound or precursor being a compound that inhibits the rate-limiting enzyme of GSH biosynthesis, γ-glutamylcysteine synthetase (GCS);
(d) a container for containing a fourth compound (iv) or a precursor thereof, said fourth compound or precursor being a compound that inhibits the enzyme responsible for the conversion of GSSG to GSH, glutathione reductase (GR); and
instructions for administration of said four compounds for treatment of cancer.

2. A kit according to claim 1 wherein:
said first compound (i) or precursor thereof is selected from the group consisting of disulfiram, diamide, diethylmalate, hydrogen peroxide precursors selected from the group consisting of ascorbic acid and dopamine, α-lipoic acid, oxidized low density lipoproteins (ox-LDLs), and a quinone selected from the group consisting of duroquinone, an ubiquinone, and β-lapachone;
said second compound (ii) or precursor thereof is selected from the group consisting of arsenic trioxide, ethacrynic acid, epothilones A and B, an α,β-unsaturated aldehyde or ketone, an unsubstituted or partially substituted quinone, an isoflavone, and a phenol;
said third compound (iii) is buthionine sulfoximine (BSO); and
said fourth compound (iv) is selected from the group consisting of 2-chloroethyl isocyanate, cyclohexyl isocyanate, 1,3-bis-(2-chloroethyl)-1-nitrosourea (BCNU or carmustine), 1-(2-chloroethyl)-3-(cyclohexyl)-1-nitrosourea, 1-(2-chloroethyl)-3-(4-trans-methylcyclohexyl)-1-nitrosourea, and 1-(2-chloroethyl)-3-(trans-4-hydroxycyclohexyl)-1-nitrosourea, ofloxacin, levofloxacin, cefepime, and cefazolin.

3. A kit according to claim 2 wherein said compound (ii) is selected from the group consisting of ethacrynic acid (EA); epothilone A and epothilone B; an α,β-unsaturated aldehyde such as cinnamaldehyde; a 4-hydroxyl-C₅-C₉-alkenal or a precursor thereof; a polyunsaturated fatty acid (PUFA); an unsubstituted or partially substituted quinone such as anthraquinone, benzoquinone, 2-methyl-benzoquinone, 2,6-dimethyl-benzoquinone, 2,5-dimethyl-benzoquinone, and 2,3,5-trimethyl-benzoquinone, □-tocopherolquinone and □-tocopherolquinone; an isoflavone such as catechin, daidzein, dicumarol, (−) epicatechin, flavopiridol, genistein, □-lapachone, myricetin and rotenone; and a phenol such as curcumin, yakuchinone A, yakuchinone B, (−) epigallocatechin-3-gallate, resveratrol, □-tocopherol, □-tocopherol, and arsenic trioxide.

4. A kit according to claim 3 wherein said first compound (i) is disulfiram; said second compound (ii) is curcumin; said third compound (iii) is buthionine sulfoximine; and said fourth compound (iv) is carmustine.

5. (canceled)

6. A kit according to claim 8, wherein said cancer is leukemia or lymphoma.

7. A kit according to claim 8, wherein said cancer is bladder, bone, brain, breast, cervical, colon, esophageal, kidney, laryngeal, liver, lung, melanoma, ovary, pancreas, prostate, rectal, skin, testicular, or uterine cancer.

8. A kit according to claim 1, wherein said cancer is a primary tumor, a secondary tumor, or metastases thereof in the same organ or in another organ.

9. A method of selectively inducing apoptosis in cancerous cells of a cancer patient, said method comprising administering to said cancer patient a pharmaceutically effective amount of a combination of 4 compounds effective to increase the redox potential of the cancerous cells to a threshold potential that induces apoptosis, while the increase in redox potential in non-cancerous cells does not induce apoptosis, wherein in said combination of 4 compounds each compound belongs to a different category selected from the group consisting of categories (i) to (iv):
(i) a compound, or a precursor thereof, that oxidizes GSH;
(ii) a compound, or a precursor thereof, that forms an adduct or a conjugate with GSH;
(iii) a compound, or a precursor thereof, that inhibits the rate-limiting enzyme of GSH biosynthesis, γ-glutamylcysteine synthetase (GCS); and
(iv) a compound, or a precursor thereof, that inhibits the enzyme responsible for the conversion of GSSG to GSH, glutathione reductase (GR).

10. The method according to claim 9, wherein the redox potential of the cancerous cells is increased and maintained continuously in the range of about −200 to −180 mV for a time such as to achieve apoptosis in the cancerous cells.

11. A method according to claim 9, wherein: said compound (i) or precursor thereof is selected from the group consisting of disulfiram, diamide, diethylmalate, hydrogen peroxide precursors selected from the group consisting of ascorbic acid and dopamine, α-lipoic acid, oxidized low density lipoproteins (ox-LDLs), and a quinone selected from the group consisting of duroquinone, an ubiquinone, and β-lapachone;
said compound (ii) or precursor thereof is selected from the group consisting of arsenic trioxide, ethacrynic acid, epothilones A and B, an α,β-unsaturated aldehyde or ketone, an unsubstituted or partially substituted quinone, an isoflavone, and a phenol;
said compound (iii) is buthionine sulfoximine (BSO); and
said compound (iv) is selected from the group consisting of 2-chloroethyl isocyanate, cyclohexyl isocyanate, 1,3-bis-(2-chloroethyl)-1-nitrosourea (BCNU or carmustine), 1-(2-chloroethyl)-3-(cyclohexyl)-1-nitrosourea, 1-(2-chloroethyl)-3-(4-trans-methylcyclohexyl)-1-nitrosourea, and 1-(2-chloroethyl)-3-(trans-4-hydroxy-cyclohexyl)-1-nitrosourea, ofloxacin, levofloxacin, cefepime, and cefazolin.

12. A method according to claim 11, wherein said compound (ii) is selected from the group consisting of ethacrynic acid (EA); epothilone A and epothilone B; an α,β-unsaturated aldehyde such as cinnamaldehyde; a 4-hydroxyl-C₅-C₉-alkenal or a precursor thereof; a polyunsaturated fatty acid (PUFA); an unsubstituted or partially substituted quinone such as anthraquinone, benzoquinone, 2-methyl-benzoquinone, 2,6-dimethyl-benzoquinone, 2,5-dimethyl-benzoquinone, and 2,3,5-trimethyl-benzoquinone, □-tocopherolquinone and □-tocopherolquinone; an isoflavone such as catechin, daidzein, dicumarol, (−) epicatechin, flavopiridol, genistein, □-lapachone, myricetin and rotenone; and a phenol such as curcumin, yakuchinone A, yakuchinone B, (−) epigallocatechin-3-gallate, resveratrol, □-tocopherol, □-tocopherol, and arsenic trioxide.

13. A method according to claim 12, wherein said compound (i) is disulfiram; said compound (ii) is curcumin; said compound (iii) is buthionine sulfoximine; and said compound (iv) is carmustine.

14. The method according to claim 9, wherein the cancer patient has a bladder, bone, brain, breast, cervical, colon, esophageal, kidney, laryngeal, liver, lung, melanoma, ovary, pancreas, prostate, rectal, skin, testicular, uterine cancer, leukemia or lymphoma.

15. A method of selectively inducing apoptosis in cancerous cells of a cancer patient, said method comprising administering to said cancer patient pharmaceutically effective amount of a combination of 4 compounds effective to prevent phosphorylation of the retinoblastoma protein (pRB) in said cancerous cells, wherein in said combination of 4 compounds each compound belongs to a different category selected from the group consisting of categories (i) to (iv):
(i) a compound, or a precursor thereof, that oxidizes GSH;
(ii) a compound, or a precursor thereof, that forms an adduct or a conjugate with GSH;
(iii) a compound, or a precursor thereof, that inhibits the rate-limiting enzyme of GSH biosynthesis, γ-glutamylcysteine synthetase (GCS); and
(iv) a compound, or a precursor thereof, that inhibits the enzyme responsible for the conversion of GSSG to GSH, glutathione reductase (GR).

16-19. (canceled)

20. A method of selectively inducing apoptosis in cancerous cells of a cancer patient, said method comprising administering to said cancer patient pharmaceutically effective amount of a combination of 4 compounds effective to prevents release of transcriptional factors from the retinoblastoma protein (pRB) in said cancerous cells, wherein in said combination of 4 compounds each compound belongs to a different category selected from the group consisting of categories (i) to (iv):
(i) a compound, or a precursor thereof, that oxidizes GSH;
(ii) a compound, or a precursor thereof, that forms an adduct or a conjugate with GSH;
(iii) a compound, or a precursor thereof, that inhibits the rate-limiting enzyme of GSH biosynthesis, γ-glutamylcysteine synthetase (GCS); and
(iv) a compound, or a precursor thereof, that inhibits the enzyme responsible for the conversion of GSSG to GSH, glutathione reductase (GR).

21-24. (canceled)

25. A method of selectively inducing apoptosis in cancerous cells of a cancer patient, said method comprising administering to said cancer patient a pharmaceutically effective amount of a combination of 4 compounds such that the effect of the four compounds on the cancer cells continuously remain for the duration of time required to arrest the cancer cells in G₁ₚₘ until apoptosis is induced whereas the normal cells in G₁ₚₘ can enter G₀ and remain unharmed, wherein each compound of the combination of 4 compounds belongs to a different category selected from the group consisting of categories (i) to (iv):
(i) a compound, or a precursor thereof, that oxidizes GSH;
(ii) a compound, or a precursor thereof, that forms an adduct or a conjugate with GSH;
(iii) a compound, or a precursor thereof, that inhibits the rate-limiting enzyme of GSH biosynthesis, γ-glutamylcysteine synthetase (GCS); and
(iv) a compound, or a precursor thereof, that inhibits the enzyme responsible for the conversion of GSSG to GSH, glutathione reductase (GR).

26. The method of claim 25 wherein said duration of time corresponds to preferably 2-5 times the normal cell-cycle time.

27. The method of claim 26 wherein said duration of time is from about 30 to about 250 hours.

28. A method of selectively inducing apoptosis in cancerous cells of a cancer patient by decreasing the [GSH]²/[GSSG] ratio such that the redox potential E in said cancerous cells is increased to or above the threshold potential, while the increase in redox potential in non-cancerous cells is such that it remains below the threshold potential, said method comprising administering to said cancer patient a pharmaceutically effective amount of a combination of 4 compounds effective for decreasing the [GSH]²/[GSSG] ratio such that the redox potential E in said cancerous cells is increased to or above the threshold potential, wherein each compound belongs to a different category selected from the group consisting of categories (i) to (iv):
(i) a compound, or a precursor thereof, that oxidizes GSH;
(ii) a compound, or a precursor thereof, that forms an adduct or a conjugate with GSH;
(iii) a compound, or a precursor thereof, that inhibits the rate-limiting enzyme of GSH biosynthesis, γ-glutamylcysteine synthetase (GCS); and
(iv) a compound, or a precursor thereof, that inhibits the enzyme responsible for the conversion of GSSG to GSH, glutathione reductase (GR).

29-39. (canceled)
